(19) United States
(12) Patent Application Publication
(10) Pub. No.: US 2018/0340701 A1
(43) Pub. Date: Nov. 29, 2018

(54) PREDICTIVE DETECTION, MINIMIZATION AND PREVENTION OF ALLERGEN, INSECT AND MOLD PARTICULATES

(71) Applicant: International Business Machines Corporation, Armonk, NY (US)

(72) Inventors: Aaron K. Baughman, Silver Springs, MD (US); Diwesh Pandey, Bangalore (IN); John P. Perrino, Hedgesville, WV (US); Todd R. Whitman, Bethany, CT (US)

(21) Appl. No.: 15/605,193

(22) Filed: May 25, 2017

(51) Int. Cl. F24F 11/00; G01N 33/00; G01N 15/06; F24F 3/16; F24F 3/14; G05B 15/02

(52) U.S. Cl. CPC F24F 11/006; G01N 33/0036; G01N 15/06; F24F 11/0017; F24F 11/0086; F24F 2011/0091; F24F 3/14; F24F 11/0079; G05B 15/02; F24F 2011/0061; F24F 2011/0023; F24F 3/1603

(57) ABSTRACT

Embodiments of the present invention provide a method, computer program product and system for detection, minimization and prevention of airborne particulates. Initially, a set of information from at least one sensor is received. The set of information comprise environmental readings. If the environmental readings exceed a threshold then a remedy to bring the set of information below the threshold is determined. Finally, the determined remedy is executed.

### BACKGROUND

The present invention relates generally to the field of environmental allergen detection and prevention, and more particularly to monitoring, detection, and prevention of allergen particulates, mold particulates and insects in enclosed areas based on cognitive and forecasting analysis.

Air quality affects an individual's health and environment. Air pollution, contaminants, and allergens are among the leading causes of chronic illness not only in the United States of America but worldwide. Many persons suffer from allergies or have hypersensitivity to airborne particles which are present in the environmental air they breathe. Air pollution, contaminants, and allergens can be caused by natural occurring or manmade chemical compounds, organic materials, and inorganic materials. Such contaminants, and particulates leading to allergies and other health issues may arise from a variety of sources, including, but not limited to: dust, pollen, mold spores, insects, pet dander, food, etc. Air pollution, contaminants, and allergens are linked to a broad range of negative health and welfare effects. Exemplary health effects include, but not limited to decreased lung function, aggravation of respiratory and cardiovascular diseases, increased asthma incidence.

### SUMMARY

According to an aspect of the present invention, there is a method that performs the following operations (not necessarily in the following order): receiving, by one or more processors, a set of information from the at least one sensor, wherein the set of information comprise environmental readings; detecting, by one or more processors, the environmental readings which exceed a threshold; and responsive to detecting the environmental readings have exceeded the threshold, determining, by one or more processors, a remedy to bring the set of information below the threshold; and executing, by one or more processors, the determined remedy.

Another embodiment of the present invention provides a computer program product for dynamic detection, minimization and prevention of allergen, insect and mold particulates in enclosed area, based on the method described above.

Another embodiment of the present invention provides a computer system for dynamic detection, minimization and prevention of allergen, insect and mold particulates in enclosed area, based on the method described above.

### BRIEF DESCRIPTION OF THE DRAWINGS

FIG. 1 is a functional block diagram illustrating a data processing environment, in accordance with an embodiment of the present invention;

FIG. 2 is a flow chart illustrating operational steps for detecting and predicting, allergies and insects and mold in order to minimize particulates in a designated area, in accordance with an embodiment of the present invention;

FIGS. 3 is a detailed flow chart illustrating another set of operational steps for detecting and predicting, allergies and insects and mold in order to minimize particulates in a designated area, in accordance with an embodiment of the present invention; and

FIG. 4 is a block diagram of the internal and external components of a computer system, in accordance with an embodiment of the present invention.

### DETAILED DESCRIPTION

Reducing the presence of unwanted particulates is generally a constant threat and costly burden on the owner of building as well as on the individuals who are susceptible for illnesses within the building. For instance, the prevention and elimination of allergen producing sources are costly and ineffective. Generally, an individual has to guess which locations within the building are potential allergen producing sources. Additionally, such actions can be dangerous and dirty due to a long exposure to chemicals; and often require frequent and repetitive actions to reduce and/or prevent allergen producing sources.

Early detection of airborne factors such as mold spores, dust mites and pollen can help remedy the issue before people begin to suffer the irritating symptoms. Similarly, knowledge, and warning of the presence of high levels of allergenic particles in the environmental air is helpful to individuals, which can provide an individual an opportunity to take precautionary actions prior to the onset of any symptoms.

Electronic devices have become an essential part of daily life. Advances in electronic technology allow for devices to communicate and exchange data. Many devices have ‘smartness’ features enabling such devices and systems to be programmed to operate in different modes. Additionally, a ‘smartness’ feature associated with many devices and systems provides for systems to run independent of human control and/or human supervision through either artificial intelligence and/or machine learning.

Further with the Internet of Things (TOT) becoming increasingly more popular, there is an emergence of new abilities to control devices and systems using applications that are installed on a wide variety of devices. The IOT is the network of physical objects (devices) containing electronic sensors, software and network connectivity, which enable the physical objects to collect and exchange data with other physical devices and/or electronic systems. The IOT, through a network infrastructure, allows objects to be sensed and controlled remotely, integrating physical objects with electronic computer systems. Specifically, an embodiment of the present invention may be associated with an aspect of IOT. For example, IOT, may utilize a one-to-one mapping and one-to-many-mapping, to establish a network of connected devices, providing an encoded data structure.

In an exemplary embodiment of the present invention, TOT may be used to monitor and control various mechanical and electrical systems used within and around a building. For example, TOT may be used to improve an individual's health by controlling lighting, heating, ventilation, air conditioning, appliances, filtration, communication systems, and autonomous robotic cleaning devices.

Embodiments of the present invention disclose dynamic and automatic techniques to forecast areas of potential issue within a building where insects can enter as well as where mold and allergies can grow and spread. Additionally, embodiments of the present invention disclose dynamic and automatic deployment of devices to prevent and/or minimize the presence of common allergen producers.

Embodiments of the present invention provide systems and methods to derive directly and/or indirectly air quality of an enclosed area, in order to determine and execute a specific action to implement upon a determination that the air quality exceeds a thresh point. Embodiments of the present invention provide systems and methods to utilize one or more sensors by receiving and analyzing environmental readings, and then accordingly determine a specific action to perform and/or specific information to provide to the user. Embodiments of the present invention determine a specific action pertinent to the user that directly and/or indirectly improves the air quality within the environment; thereafter perform the determined action (e.g., via motorized structures such as networked robots controlled via a processor) for implementing the solution. Therefore, embodiments of the present invention may allow for individuals to reduce using medication both pre and post exposure to allergens, mold and insects commonly found in low air quality environments. For instance, being exposed to less allergens, mold and insects may minimize and reduce the amount of medication an individual takes to alleviate symptoms associated with exposure. Further, embodiments of the present invention may allow for individuals to move about more freely within an environment, as each individual is not forced, due to health reasons based on poor air quality, to avoid specific locations. Since, individuals cannot always plan to avoid every possible airborne allergen in a particular environment, embodiments of the present invention provide a significant increase to the control and performance of maintaining high quality air within an environment. For example, embodiments of the present invention provide a user the ability to detect and clean the air prior to airborne particulate levels reaching a health hazard. Similarly, embodiments of the present invention provide a user the ability to be proactive and dispatch robotic instruments to clean specific areas of concern. Furthermore, embodiments of the present invention provide a user the ability to utilize augmented reality to directly visualize an area of concern and implement one or more remedies manually

Embodiments of the present invention recognize the need for more effective detection and elimination of allergens, mold and insect particulates within an environment. Improving the air quality by detecting and illuminating unwanted particulates without the direct instructions and/or actions from a human significantly improves the internal air quality of a building. For example, since an embodiment of the present invention can forecast and predict areas of concern, remedial action may be taken prior to any degradation of the air quality. Further, autonomous cleaning devices, may be dispatched to provide specific remedial action at locations where it is unsafe and/or inaccessible to a human. Since embodiments of the present invention provide for preventative action, the overall air quality within the environment may increase thereby reducing a wide variety of health issues individuals may suffer from.

Embodiments of the present invention provide a system and method that may through analytic analysis, generate a model of the environment to highlight one or more areas of concern to a user. Embodiments of the present invention provide a system and method which through machine learning modules and/or artificial intelligence, generate a model of the environment to highlight one or more areas of concern to a user. All of which thereby allowing a user to precisely perform corrective action and/or to automatically perform corrective action.

Specifically, embodiments of the present invention provide systems and methods to detect and analyze the air quality of a given environment in order to determine whether the air is within a harmless level. Further, embodiments of the present invention provide systems and methods to determine what action can and should be taken upon determining the air quality in a given environment is not within a normal limit. Additionally, embodiments of the present invention provide systems and methods to execute and perform the determined actions to improve the air quality in a given environment. Moreover, embodiments of the present invention provide systems and methods to predict and perform remedies to areas of concern within a building thereby preemptively improving the air quality within a given environment.

Embodiments of the present invention utilize one or more sensors allowing user(s) to control the environments to a certain extent and increase awareness of the pollutants around them. Generally, if a user is more aware of the contents of the environment, the user can change their routines and habits in order to be less effected by air pollution and allergies. Additionally, a system may utilize sensors to detect air contaminants, and based on a set of results perform one or more actions to reduce and/or eliminate contaminants to a safe level. This can help increase the quality of life and overall health of people at risk. For example, if one location is determined to be unsafe due to the level of pollutants, an embodiment of the present invention may execute one or more remedies to reduce the contaminants. Additionally, embodiments of the present invention can establish safe levels of environmental contaminants based on the indeed user, i.e., infants or the elderly, and anyone else that is at risk.

Embodiments of the present invention disclose a system and method to forecast areas where insects can enter, mold could grow and allergens spread throughout the home. Common detectors such as CO2, images of color, and moisture detectors will transmit data, thereby allowing embodiments of the present invention to identify, analyze, predict, provide a remedy, and prevent detected issues. Specifically, devices may be deployed around the structure to inspect for cracks or crevices. Heat maps will find where there are air gaps in the structure. The deployed devices may spray insecticide and mold inhibitors in problem areas as well as fill in air gaps, if possible. If performing these functions is not possible, the deployed devices will mark the location of the gap on a layer that can be seen with augmented reality.

The cognitive home environmental change detection system has the ability to provide the following improvements to cognitive home environment change detection systems including but not limited to: (i) monitor changes to environmental factors, and (ii) alert and react when thresholds have been exceeded. Stated differently, when monitoring changes, embodiments of the present invention can account for static and/or dynamic behaviors of the environment. For example, when thresholds have been exceeded during forecasting and simulation of the environment into a future state, one or more remote controlled devices are deployed to remedy. The remote controlled devices may perform the following remedies: (i) closing air gaps, (ii) spraying insecticide, and (iii) depositing mold inhibitors within trouble locations. The remote controlled devices may perform a closer inspection of the cognitive structure to put the status within a map on augmented reality. Thereafter, a user may load the layer within their augmented reality display to view the probability of a troubling area emerging. Additionally, the remote controlled devices may move devices around the home or suggest additional devices should be placed to gather more accurate information.

Embodiments of the present invention enhance detection and prevention of allergenic particles in the environmental air. Further, embodiments of the present invention may predict areas of concern within a building and thereby preemptively resolve any issues prior to the air quality deteriorating. Techniques for providing improved air quality within a building may include detection and controlling various objects within an environment. For example, upon detection of an area of concern, embodiments of the present invention may dispatch one or more autonomous cleaning devices to decontaminate and clean the identified location.

Embodiments of the present invention provide a system to predict and forecast ecologies of a certain area and generate a prescriptive plan to prevent negative environment factors in a cognitive structure.

Embodiments of the present invention may provide one or more devices to fill in airgaps and spray insecticide for prevention of allergen, mold and negative environmental factors for humans. Embodiments of the present invention provide a system of remote controlled and/or autonomous devices which can move or add sensors to the cognitive structure to increase the precision of forecasting.

Embodiments of the present invention may use augmented reality by marking troubled areas with probabilities that are viewable by humans.

Embodiments of the present invention utilize individualized allergy, insect, and treatment profiles based on a specific user, a set of users, as well as ones location in the world.

Embodiments of the present invention provide a system and method that may self-learn, based on objects within an environment as well as recurring areas of concern.

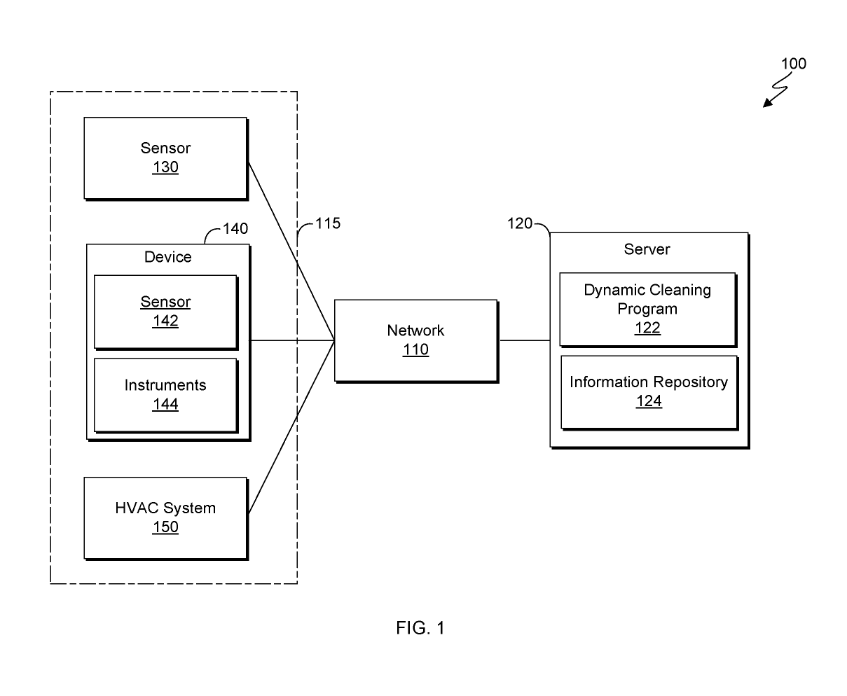

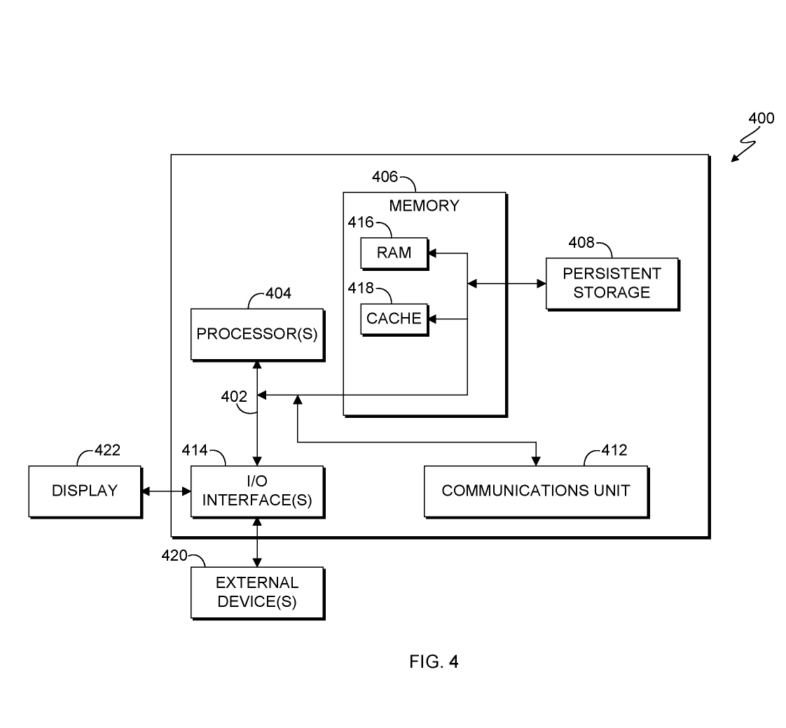

The present invention will now be described in detail with reference to the Figures. FIG. 1 is a functional block diagram illustrating a data processing environment (“environment”), generally designated 100, in accordance with an embodiment of the present invention. FIG. 1 provides only an illustration of one embodiment and does not imply any limitations with regard to the environments in which different embodiments may be implemented. Many modifications to the depicted environment may be made by those skilled in the art without departing from the scope of the invention, as recited by the claims. In this exemplary embodiment, environment 100 includes sensor 130, device 140, HVAC System 150 (within location 115) and server 120 interconnected over network 110. Device 140, and server 120 may include internal and external hardware components, as depicted and described in further detail with respect to FIG. 4.

Network 110 may be a computer network with a small geographic scope. Computer networks with a small geographic scope range from Near Field Communication (NFC) to Local Area Networks (LANs). A computer network with a small geographic scope typically does not have a connection to the Internet or other remote networks. In an alternative embodiment, network 110 is not intended to be limited to a small geographic scope, rather network 110 may include a larger networking environment.

Network 110 may be a local area network (“LAN”), a wide area network (“WAN”), such as the Internet, the public switched telephone network (“PSTN”), a mobile data network (e.g., wireless Internet provided by a third or fourth generation of mobile phone mobile communication), a private branch exchange (“PBX”), any combination thereof, or any combination of connections and protocols that will support communications between server 120, sensor 130, device 140 and HVAC system 150.

Network 110 architecture may include one or more information distribution network(s) of any type(s), such as, cable, fiber, satellite, telephone, cellular, wireless, etc., and as such, may be configured to have one or more communication channels. In another embodiment, network 110 may represent a “cloud” of computers interconnected by one or more networks, where network 110 is a computing system utilizing clustered computers and components to act as a single pool of seamless resources when accessed.

The various aspects of network 110 are not limited to radio frequency wireless communications; rather, communication may be accomplished via any known mediums in the art, including but not limited to, acoustic mediums, and optical mediums, such as, visible or infrared light. For example, data exchanged between devices, may be transmitted via infrared data links using well known technologies, such as infrared transceivers included in some mobile device models.

Location 115 represents any structure which is enclosed and has treated air such as though an air conditioning system. Location 115 may be a standalone room or a portion of a larger building or an entire building. For example, location 115 may represent a residential home, residential apartment complex, an office complex, industrial building and/or commercial building, etc. Location 115 may represent one or more zones within an establishment. For example a zone may be defined as a portion of a room, a single room, multiple rooms, an entire floor of a building, a section of a building, and/or multiple buildings within a multi-building complex. Regardless of the size of each zone, location 115 defines the proximate area to which dynamic cleaning program 122 monitors and dynamically eliminates mold, allergies and insect particulates. In this embodiment, location 115 includes sensor 130, device 140 and HVAC system 150. For illustrative purposes, FIG. 1 shows location 115 as having a single sensor, device, and HVAC system. However, it should be understood that location 115 can include any number and combination of sensors, devices, and HVAC systems.

Sensor 130 senses, detects and/or measures various atmospheric, air quality and environmental aspects within location 115. Environment 100 depicts only a single sensor, sensor 130, however, it should be understood that multiple sensors (i.e., multiple units of sensor 130) can be included. Sensor 130 may detect particulates found in the air which can have adverse effects on a humans overall health. Often humans cannot see or detect slight changes to one's environment therefore, sensor 130 can provide a significant amount of information. Exemplary sensors may include: (i) temperature sensors; (ii) humidity sensors (to detect whether moisture should be removed or added); (iii) barometric pressure sensor; (iv) mold sensors, (v) radon sensors; (vi) Co2 sensor; and (vii) carbon dioxide sensor; (viii) air quality sensors (i.e., fine particulate matter sensor; course particulate matter sensor; volatile organic compound (toxins))

Sensor 130 may detect the air quality within location 115. Air quality is a term used to relate how much allergens, and/or pollution is present within the air of location 115. For example, good air quality means there is less particulates which can cause harm to a user, whereas poor air quality means there is more particulates in the air which may cause harm to a user. Good air quality may be measured in parts per million and dependent on the particulates detected. For example, 0.2 parts per million of carbon monoxide is considered good air quality; however as carbon monoxide reach 70 parts per million, results in noticeable symptoms. In another example, mercury levels in the air exceeding 0.05 mg/m³ results in poor air quality. Sensor 130 may detect a variety of pollutants for example, ozone (O₃), sulfur dioxide (SO₂), oxides of nitrogen, carbon monoxide (CO), lead (Pb), ammonia (NH₃), volatile organic compounds (VOCs), mercury (Hg), as well as other toxic air pollutants. Sensor 130 may detect a variety of allergens which, are common organic particles, and mold spores which commonly cause health issues for humans.

Specifically, sensor 130 represents one or more sensor or sensor array used to detect particulates within location 115. Sensor 130 represents one or more devices that detect and monitor the presence of air pollution, allergies, mold, and insects within location 115. Sensor 130 may represent a sensor specialized to detect a variety of environmental factors such as ozone, particulate matter, carbon monoxide, sulfur dioxide, nitrous oxide, allergens, mold particulates, evidence of insects, etc.

Sensor 130 may take instantaneous air quality detections and/or take measurements based on an average of particulates detected over a given time period For example, sensor 130 may take measurements at predetermined intervals or upon a request by a user, thereby providing an instantaneous reading as compared to a previous given time interval.

Sensor 130 may be a network air sensor used to measure particulate matter variations throughout one or more locations, e.g., location 115. Sensor 130 may monitor air quality of one or more individuals while performing normal activities within location 115. For example, if location 115 is a hospital, where it is more desirable to improve air quality, then sensor 130 may have a threshold higher/lower than what is typically accepted as compared to if location 115 is a residential home.

Sensor 130 types and performance may differ according to the application. Thus, the quality of a measurement needed is dictated by the basic performance of the sensor, the means by which the sensor is operated, and the way dynamic cleaning program 122 analyzes the results.

Sensor 130 may include a variety of different sensor types. For example, sensor 130 may include pressure sensors, audio sensors, water sensors, particulate counter sensors, pesticide sensors, explosive gas sensors, emission sensors, biological agent sensors, chemical agent sensors, NO₂ sensors, CO sensors, and heat sensors.

Sensor 130 may include sensing techniques including, but not limited to processing of signals representing geographic locations in order to detect and/or infer one or more conditions under which dynamic cleaning program 122 may determine pertinent information to display on to a user. It is noted that a geographic location may include any type of bonded area of interest meaningful to a user and/or dynamic cleaning program 122. A geographic location may be distinguished by one or more location 115 via a physical or virtual boundary line, which may or may not correspond to and/or overlap with a physical location and/or structure (e.g., sections of large commercial building, individual shops in a large mall, sections of a particular shop, a particular room of a residence, a cubical in an office building, predesignated HVAC zones, etc.). In some exemplary geographic locations, a user may define one or more zones within a geographic area. Zones may be of a different size and even overlap with one or more zones.

Device 140 represents one or more remote controlled robot type autonomous devices, capable of being self-proposed. Each device 140 may have one or more sensor(s) 142 and a set of instruments 144. Specifically, device 140 is self-contained, intended to autonomously navigate to perform corrective actions to an area of concern. Device 140 is utilized to pinpoint and eliminate any allergen particulates, mold particulates and/or insects detected within location 115. Device 140 may operate autonomously or be controlled by dynamic cleaning program 122. Device 140 has a mobility function, allowing device 140 to travel to the area of concern, as determined by dynamic cleaning program 122. For example, device 140 may travel to the area of concern by either flying, and/or driving on wheels or tracks. Device 140 may have the ability to avoid and/or move obstacles in its path in order to access the area of concern. Additionally, device 140 may be utilized to access (i) areas which are harmful to humans or (ii) a location a human cannot reach.

In an embodiment device 140 may include one or more objects including, but not limited to: (i) remote controlled and/or autonomous device which can fly allowing travel to area(s) of concern; (ii) remote controlled and/or autonomous device with tracks allowing travel to area(s) of concern.

Regardless of the type method by which device 140 travels and navigates within location 115, in an embodiment, device 140 may have the ability to receive information directly from sensors and act on that information. In an embodiment, device 140 may have the ability to receive information from dynamic cleaning program 122 which analyzes information from sensor 130 and directs device 140 to act. In an embodiment, device 140 may have the ability to activate filters or other miscellaneous devices (i.e. dehumidifiers). In an embodiment, device 140 may have the ability to take some limited mechanical actions to rectify an issue (i.e. spray insecticide or sealant). In an embodiment, device 140 may have the ability to record actions taken and whether those actions were successful or need further follow-up action(s) to be taken. In an embodiment, device 140 may have the ability to interact with Augmented Reality.

Device 140 may also have a communication port (not shown), allowing it to communicate with dynamic cleaning program 122. Device 140 may communicate with dynamic cleaning program 122 through wired and/or wireless communication methods known by those skilled in the art. For example, device 140 may communicate with dynamic cleaning program 122 through either networks with a small geographic scope range from Near Field Communication (NFC) to Local Area Networks (LANs). For instance, device 140 may communicate with dynamic cleaning program 122 through a wireless personal area network (“WPAN”), a private network carried over wireless network technologies such as Bluetooth® or peer-to-peer communications over a wireless LAN (Note: the term “Bluetooth” is a registered trademark of Bluetooth SIG, Inc. and may be subject to trademark rights in various jurisdictions throughout the world and are used here only in reference to the products or services properly denominated by the marks to the extent that such trademark rights may exist).

Sensor 142, may be similar to those of sensor 130, thereby providing device 140 the ability to detect and pinpoint the exact area of concern. Additionally, and/or alternatively, sensor 142 may have different capabilities and sizes dependent on their intended purpose.

In this embodiment, device 140 includes instruments 144. In other embodiments, device 140 may include one or more instruments 144 in order to eliminate and/or reduce allergen, insect and mold particulates. Instruments 144 may be one or more insecticides which device 140 will discharge over the area of concern as determined by dynamic cleaning program 122, to prevent and/or eliminate any detected insects. Instruments 144 may be one or more mold inhibitors, which will discharge over the area of concern as determined by dynamic cleaning program 122, to prevent and/or eliminate the presence of mold. Instruments 144 may be one or more sealants, which will discharge over the area of concern as determined by dynamic cleaning program 122, to seal off an area which is letting in allergens, mold and/or insects.

Device 140 may utilize sensor 142 to accurately execute a specified remedy once device 140 is within the area of concern of location 115. For example, if dynamic cleaning program 122 determines that a particular area of location 115 has an increased presence of particulates, then dynamic cleaning program 122 may dispatch device 140 to the area of concern. Thereafter, device 140, through sensor 142, may pinpoint and accurately navigate to the location causing the problem and perform one or more remedies based on dynamic cleaning program 122 instructions. For instance, if there is a crack in the wall, device 140 may seal the wall using instruments 144. For another example, device 140 may discharge pesticides to eliminate and/or prevent unwanted insects.

In an embodiment device 140 may be programmed for a fixed routine providing various operations (i.e., start, stop, clean, dispense pesticides, etc.). For example, device 140 may be programed to run at a predetermined time interval. In another example, devices may also be started on an occurrence of a particular event. For instance, sensors may continually scan an area and engage a particular item upon detecting an issue with the air quality.

HVAC system 150 may be a standalone system or a tie in node and provides dynamic cleaning program 122 access to the buildings existing HVAC systems, in order to leverage various functionalities of an existing HVAC system. HVAC system 150 accesses the existing heating, ventilation, air conditioning (hereinafter “HVAC”) system, to filter, and/or circulate air within a building may be imitated based on either predetermined time or responsive to a specific event occurring, such as a detection of poor air quality. HVAC system 150, may control a plurality of filtration systems to filter out one or more particulates detected by sensor 130. For example, upon a detection of allergies, HVAC system 150 may activate one or more filters designed to remove allergies from the air. In another example, upon detection of mold, HVAC system 150 may remove moisture (humidity) from the air, thereby prohibiting the growth of mold, within location 115.

For example, by leveraging the existing HVAC system, dynamic cleaning program 122, may, if the level of particulates increases above a desired threshold, engage filtration systems and/or flush the buildings air supply.

In an embodiment HVAC system 150 may be programmed for a fixed routine providing various operations (i.e., start, stop, circulate air, filter air, etc.). For example, HVAC system 150 may be programed to run at a predetermined time interval, in addition to running upon detecting particulates above a threshold level.

In this embodiment, server 120 includes dynamic cleaning program 122 and information repository 124 and is responsible for increasing control and performance of maintaining high quality air within an environment. In an exemplary embodiment, server 120 is a server computer. Server 120 can be desktop computers, laptop computers, specialized computer servers, or any other computer system known in the art. Server 120 may be located within location 115 or remotely connected through network 110.

In certain embodiments, server 120 represents computer systems utilizing clustered computers and components to act as a single pool of seamless resources when accessed through network 110. For example, such embodiments may be used in data center, cloud computing, storage area network (SAN), and network attached storage (NAS) applications. In certain embodiments, server 120 represents virtual machines. In other embodiments, server 120 may be a management server, a web server or any other electronic device capable of receiving and sending data. In another embodiment, server 120 may represent a server computing system utilizing multiple computers as a server system, such as in a cloud computing environment. In general, server 120 is representative of any electronic devices, or combination of electronic devices, capable of executing machine-readable program instructions, as described in greater detail with regard to FIG. 4. Server 120 contains dynamic cleaning program 122, and information repository 124.

Dynamic cleaning program 122 may be located as depicted in server 120; however in alternative embodiments (not shown) dynamic cleaning program 122 may be located on a remote cloud. Dynamic cleaning program 122 may be stored in a persistent storage component (not depicted) for execution and/or access by one or more processor(s) via one or more memories (for more detail refer to FIG. 4). While depicted on server 120, in the exemplary embodiment, dynamic cleaning program 122 may be on a remote server or a “cloud” of computers interconnected by one or more networks utilizing clustered computers and components to act as a single pool of seamless resources, accessible to dynamic cleaning program via network 110.

Dynamic cleaning program 122 may be interconnected and associated with multiple locations (i.e., location 115a, location 115B, through location 115n, not shown in FIG. 1). For instance, location 115a, location 115B, through location 115n (not shown in FIG. 1) may be independent locations where dynamic cleaning program 122 may manage multiple independent locations. In another instance, location 115a, location 115B, through location 115n (not shown in FIG. 1) may be a specific zone within a larger single location, such that each zone is one or more rooms within a single larger location. Thereby, dynamic cleaning program 122 maintains the ability to control and alter the air quality in specific zones of location 115 while not affecting other zones of location 115.

Dynamic cleaning program 122 may have access to location services and/or environmental services. For example, location services may include global positioning services (GPS) and other location determining services known in the art. Dynamic cleaning program 122, based on the global location of location 115, may have the ability to look up average environmental factors for the area and/or on demand environmental factors. For instance, if the geographic area location 115 is located within, is known for very wet season, then dynamic cleaning program 122 may proactively dehumidify the air and dispense anti mold and insect precautions. In another instance, if an on demand warning is received, which specifies a swarm of locusts, dynamic cleaning program 122 may proactively dispatch one or more remedies to prevent the locust from entering the building (i.e., increase the air pressure within location 115, discharge insect repellant, seal any areas of concern of the building, etc.). In another instance, if an on demand warning is received, which specifies a high allergen warning, dynamic cleaning program 122 may proactively engage filters to prevent the significant presence of allergens within the building.

Further, dynamic cleaning program 122 may be capable of determining proper air quality within location 115, such that, air quality may be dependent on specific location, i.e., an individual's home, place of employment, car, child's school, etc. Similarly, dynamic cleaning program 122 may be capable of determining proper air quality within location 115 based on the geographic location of location 115. In the various embodiments of the present invention, dynamic cleaning program 122 receives various data from sensor 130 as well as geographical location of location 115, and determines where to set each threshold based on detected and received data.

Dynamic cleaning program 122 may utilize one or more thresholds for various environmental particulates. Stated differently, dynamic cleaning program 122 may analyze the air quality and place the air quality sample into one or more levels. In an embodiment, dynamic cleaning program 122 may have one or more thresholds predefined. In an embodiment, dynamic cleaning program 122 may determine one or more thresholds based on the building purpose, and/or geographic location of location 115. In an embodiment, dynamic cleaning program 122 may automatically alter one or more thresholds based on received environmental warnings and/or learned patterns. Among other factors, dynamic cleaning program 122 may have each threshold level predefined based on geographic location and the intended use of location 115 Dynamic cleaning program 122 may have each level predefined based on geographic location, the intended use of location 115, among other factors.

Dynamic cleaning program 122 may include one or more of the following thresholds: (i) allergens; (ii) mold; (iii) insects; (iv) humidity; (v) temperature; (vi) undesirable airborne particulates; and (vii) pests (i.e., rodents). The threshold levels may be established on the type and/or quantity of particulates found within location 115. Exemplary threshold levels may include: (i) good; (ii) moderate; (iii) unhealthy for sensitive groups; (iv) unhealthy; and (v) hazardous. Each threshold may be on a different scale dependent on the particulate(s) detected. Therefore, depending on the identified threshold level, dynamic cleaning program 122 may perform different actions to improve the air quality within location 115.

In addition to accessing the existing HVAC system, dynamic cleaning program 122 may also deploy and/or control a set of device 140, (e.g., remote controlled robots) which can accurately and efficiently, pinpoint and perform corrective action to an area of concern as detected by dynamic cleaning program 122. Dynamic cleaning program 122 may also be in communication with each device 140 in order to derive an enhanced analysis of an area of concern.

Dynamic cleaning program 122 may forecast areas where insects, pests (e.g., rodents) can enter. For example, based on received information from sensor 130, dynamic cleaning program 122 may determine an area of concern and/or interest. Dynamic cleaning program 122 may forecast areas where mold can grow. For example, based on received information from sensor 130, dynamic cleaning program 122 may determine an area of concern. Dynamic cleaning program 122 may forecast areas where allergens can enter location 115 and/or accumulate. For example, based on received information from sensor 130, dynamic cleaning program 122 may determine an area of concern.

Dynamic cleaning program 122 may provide a user with access to augmented reality to inspect an area of concern. Specifically, dynamic cleaning program 122 may generate an interactive model of the environment on an augmented reality system to display on a graphical user interface (GUI) a specific area of concern. For example, dynamic cleaning program 122 may provide a user an overlay of location 115 and highlight a specific area of concern. For instance, if user wears virtual reality glasses, dynamic cleaning program 122 may generate a display, and overlay the display on a portion of the virtual reality glasses, and highlight an area of concern, in real time, to user. In another example, dynamic cleaning program 122 may display on a monitor an area of concern within location 115.

Dynamic cleaning program 122 can, in response to a user selection update the interactive model. For example, dynamic cleaning program can display on an area flagged as an area of concern a selectable list of options to remedy or improve the area of concern (e.g., display a selectable list of options to remedy allergen levels in a specific area of concern). Responsive to a user selecting an option from the list of selectable list of options, dynamic cleaning program 122 can update the display to show a decrease in concern to the area. For example, in instances where a color scheme is used (e.g., green, yellow, red denoting acceptable, borderline, and unacceptable levels of allergens), dynamic cleaning program 122 can update the display to show the area of concern switch from red to green. In other instances, dynamic cleaning program 122 can increase or decrease a perimeter associated with an area of concern in response to a user selection in combination with updating a color scheme. For example, a user may select to deploy devices to reduce an area of concern designated as red (a color associated with non-acceptable levels of allergens) that reflects an area of 36 inches by 40 inches. In response to a user selection, and after the particular remedy is performed, dynamic cleaning program 122 can update the display on the user device to show that a portion of the 36 in. by 40 in. area is now highlighted in green (a color associated with acceptable levels of allergens) while another portion is highlighted in yellow (a color associated with non-acceptable levels of allergens).

In an embodiment dynamic cleaning program 122 may suggest to user additional device 140 needed to provide proper remedy to a specific issue. Additionally, in an embodiment, dynamic cleaning program 122 may suggest additional sensor 130 (and/or sensor 142) types and locations in order to gather more accurate information.

Information repository 124 stores information received from sensor 130. In other embodiments, information repository 124 may store information from one or more other components of environment 100. In this embodiment, information repository 124 may include any suitable volatile or non-volatile computer readable storage media, and may include random access memory (RAM) and cache memory (not depicted in FIG. 1). Alternatively, or in addition to a magnetic hard disk drive, the persistent storage component can include a solid state hard drive, a semiconductor storage device, read-only memory (ROM), erasable programmable read-only memory (EPROM), flash memory, or any other computer readable storage media that is capable of storing program instructions or digital information. While information repository 124 is depicted on server 120 it is noted that Information repository 124, may be on a remote server or a “cloud” of computers interconnected by one or more networks utilizing clustered computers and components to act as a single pool of seamless resources, accessible to dynamic cleaning program 124 via network 110. Information repository 124 may be implemented using any architecture known in the art such as, for example, a relational database, an object-oriented database, and/or one or more tables. Information repository 124 stores actual, modeled, predicted, or otherwise derived patterns of environmental air quality based on received sensor data. For example, information repository 124 stores all information received from sensor 130. Information repository 124 may contain lookup tables, databases, charts, graphs, functions, equations, and the like that dynamic cleaning program 122 may access to both maintain a specific parameter as well as manipulate various parameters within the location 115. Information stored in information repository 124 may include: various geographical locations, specific actions linked to a various detected issue, a set of rules, learned air quality patterns, and the like.

Information repository 124 may contain information which dynamic cleaning program 122 may leverage in determining specific actions to perform based on a variety of sensor 130 readings. Similarly, information repository may contain various environmental factors which may utilize dynamic cleaning program 122 in determining one or more thresholds and/or particular remedies. Information repository 124 may contain historic data from sensor readings and from previous determinations thereby providing dynamic cleaning program 122 any requisite information to predict an increase of a certain particulate. For example, allergens in the spring season are typically worse than allergens in during winter, therefore dynamic cleaning program 122 can modify its air quality analysis based on real time input.

In an embodiment information repository 124 may contain sensor 130 and/or sensor 142 readings in order to forecast and predict when common issues arise. In an embodiment information repository 124 may contain atmospheric conditions (i.e., weather conditions). For example, dynamic cleaning program 122 may cross reference information contained in information repository 124 to derive a pattern, thereby predicting issues before a threshold is met. In this example, dynamic cleaning program 122 may execute one or more remedies to prevent an issue from arising.

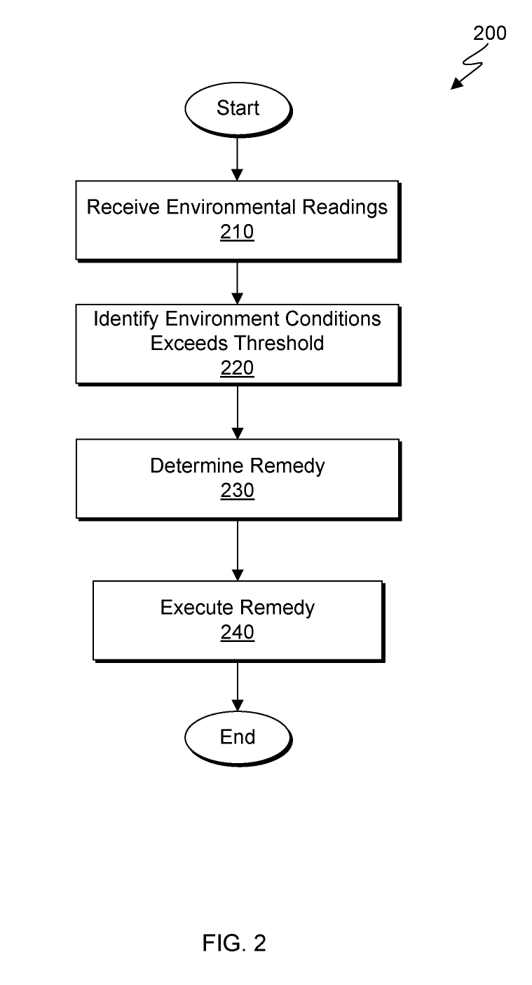

Reference is now made to FIG. 2. FIG. 2 is a flow chart illustrating operational steps for detecting and predicting, allergies insects, pests, hazardous chemicals and mold in order to minimize particulates in a designated area, in accordance with an embodiment of the present invention.

In step 210, dynamic cleaning program 122 receives environmental readings from sensor 130. In an embodiment, dynamic cleaning program 122 may autonomously activate sensor 130. In another embodiment, dynamic cleaning program 122 may instruct senor 130 to take readings at predefined schedules and transmit the readings to dynamic cleaning program 122. In an embodiment, dynamic cleaning program 122 may instruct sensor 130 to continually record and transmit environmental readings to dynamic cleaning program 122. In an embodiment, a user can instruct dynamic cleaning program 122 to receive environmental readings from sensor 130, on demand. In an embodiment, sensors 130 may continually sense for the presence of certain particulates. In an embodiment, dynamic cleaning program 122 receives environmental readings from both sensor 130 and sensor 142. In yet another embodiment, dynamic cleaning program 122 can receive environmental readings from one or more other components of environment 100.

In another embodiment, dynamic cleaning program 122 may direct sensor 130 to only be engaged upon certain conditions. For example, sensors 130 may engage upon detecting a human presence in the vicinity of location 115. Alternatively, sensors 130 may engage upon no human presence in location 115. In alternative embodiments, location 115 may be subdivided into designated zones, whereby each zone's array of sensors are controlled individually. Embodiments of the present invention may utilize a manual override whereby a user can manually engage or disengage sensor 130.

In step 220, dynamic cleaning program 122 compares the received environmental readings from sensor 130 to one or more thresholds. In an embodiment, dynamic cleaning program 122 may determine an appropriate air quality level and assign a threshold to it. In alternative embodiments, dynamic cleaning program 122 may have a specific air quality level pre-defined. In an embodiment a threshold may be derived by dynamic cleaning program 122.

In an embodiment, dynamic cleaning program 122 may adjust one or more thresholds, based on a derived pattern and/or predictive forecasting. Further, in an embodiment, dynamic cleaning program 122 may analyze the environmental readings and create a forecasting of future air quality, and adjust thresholds as necessary to maintain and/or improve air quality. Similarly, in an embodiment, dynamic cleaning program 122 may create a forecasting of future air quality, based on accessing weather conditions and comparing the current conditions to that of historical data to predict air quality. For example, dynamic cleaning program 122 may access various weather patterns and compare such patterns to historical data. Upon comparing location 115 weather patterns near location 115, dynamic cleaning program 122 may predict common issues and adjust a threshold, accordingly. For instance, if dynamic cleaning program 122 makes a connection between wet weather and an increase in insects, dynamic cleaning program 122, may lower the threshold, thereby making it more likely to perform a remedy. Thereby, dynamic cleaning program 122 may adjust each threshold according to outside conditions.

In an exemplary embodiment, the threshold may continually change based on a derived forecasting for location 115. For instance, if it is the wet season, the threshold may be lowered to detect more insect and/or mold presence. In another example, if it is the dry season, the threshold for mold may increase as it is less likely mold particulates will be present within location 115.

Dynamic cleaning program 122 may compare environment conditions to one or more thresholds. A threshold may be based on a known quantity, ratio, percentage, of: (i) insects; (ii) allergens; (iii) mold; (iv) contaminants, and the like.

If in step 220, dynamic cleaning program 122 identifies the environmental conditions (received in step 210) are within a given threshold, then dynamic cleaning program 122 may continue monitoring until such time, end, or restart at step 210.

Upon dynamic cleaning program 122 identifies the environmental conditions exceed a threshold, then in step 230, dynamic cleaning program 122 determines one or more specific remedies in order to decrease, prevent and/or eliminate specific particulate(s) at issue. Exemplary remedies may include accessing and adjusting the HVAC system through HVAC system150: (i) engaging the HVAC filtration system; (ii) engaging the HVAC circulation system; (iii) increasing the building pressure; (iv) seal the building; (v) engaging humidifiers/dehumidifiers (vi) engaging radon fans; (vii) engaging multi-gas air purification system. Exemplary remedies may also include dispatching and directing one or more device 140 to: (i) discharge anti-mold; (ii) discharge pesticides; (iii) discharge sealant; (iv) cleanup (vacuum) pet dander; and (v) dispense pest (rodent) elimination technique (i.e., emit sound wave to drive pest away, dispense pesticide designed to eliminate, and electricity).

In step 240, dynamic cleaning program 122 executes the one or more determined remedies. For example, dynamic cleaning program 122, in step 230, may determine to deploy a device to discharge an anti-mold agent at a particular location (i.e., “the location at issue”). In another example, dynamic cleaning program 122, in step 230, may determine to deploy a device to discharge a specific type of pesticide along an area where specific insects were detected. In another example, dynamic cleaning program 122, in step 230, may determine to deploy a device, to seal a particular area where insects and/or allergens were entering the location at issue.

In another example, dynamic cleaning program 122, in step 230, may determine to deploy a device to perform a closer inspection of a certain area, through sensor 142. For instance, dynamic cleaning program 122 may deploy a device for a closer inspection if it is unable to determine a specific cause of an issue from sensor 130. Thereafter, dynamic cleaning program 122 may determine a remedy per step 230. Additionally, and/or alternatively, dynamic cleaning program 122 may present the issue to a user using augmented reality, highlighting the area of concern; whereby a user can inspect the one or more areas at issue which dynamic cleaning program 122 is unable to resolve.

In another example, dynamic cleaning program 122 may access the building's HVAC system in order to execute the determined remedy. For example, dynamic cleaning program 122 may: (i) engage the HVAC filtration system; (ii) engage the HVAC circulation system; (iii) increase the building pressure, etc. The filtration system can comprise high efficiency particulate air (HEPA) filters, UV filtration, carbon filters, ionizers, ozone generators, antibacterial filters, germicidal filters, and the like. Similarly, by increasing the buildings internal pressure, may inhibit insects and particles from entering the building, as such items must overcome the pressure difference.

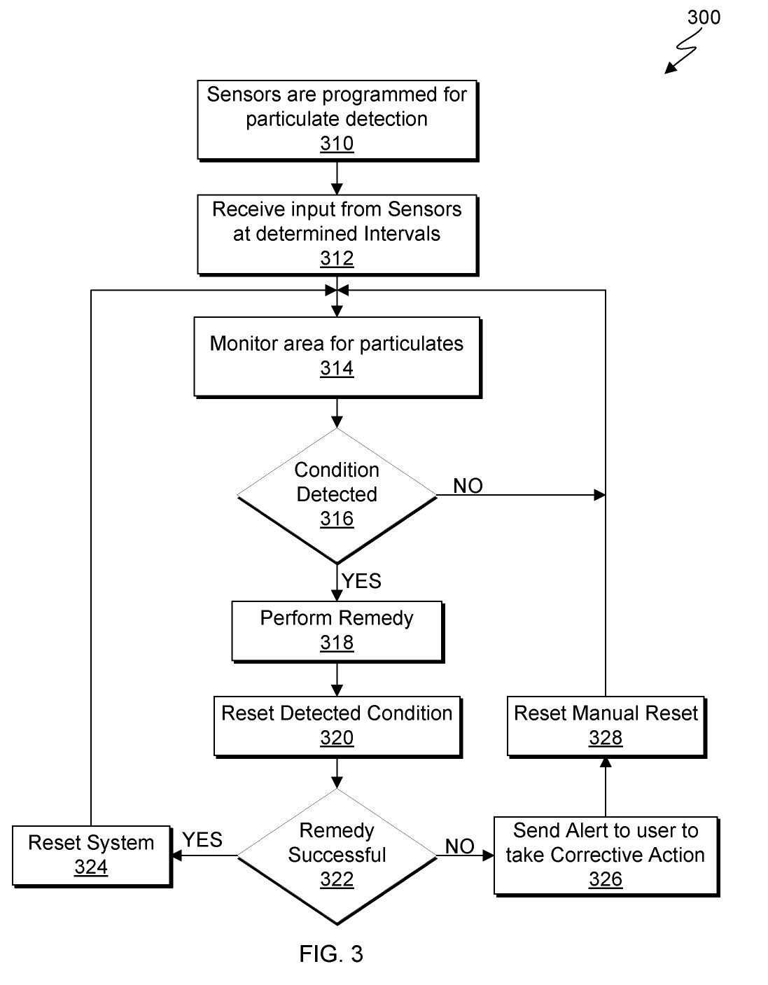

Reference is now made to FIG. 3. FIG. 3 depicts flow chart 300 illustrating another set of operational steps for detecting and predicting allergies, insects and mold in order to minimize particulates in a designated area, in accordance with an embodiment of the present invention.

Step 310 and step 312 are preliminary steps, in which one or more sensors/monitors are placed within location 115 and programed to detect one or more particulates. Each sensor/monitor can detect one or more items such as, allergens, mold, insects, carbon dioxide, heat sensors, etc. In step 312, dynamic cleaning program 122 requests and then receives the readings from each sensor/monitor. Each request by dynamic cleaning program 122 of sensor may be directed based on predetermined intervals, predetermined events, and/or a request by a user.

In step 314, dynamic cleaning program 122 monitors location 115 for particulates. In this embodiment, dynamic cleaning program 122 monitors location 115 for particulates by engaging sensor 130. In this embodiment, dynamic cleaning program 122 engages sensor 130 by transmitting instructions to monitor and transmit feedback from sensor 130 to dynamic cleaning program 122. In other embodiments, dynamic cleaning program 122 queries sensor 130 for information according to a set time interval. In yet other embodiment, dynamic cleaning program 122 can derive particulate information from one or more other components of environment 100.

In step 316 dynamic cleaning program 122 determines if a particulate is detected, and if the detected particulate exceeds a given conditional threshold. If the detected particulate is within an appropriate (normal) level, then dynamic cleaning program 122 continues to monitor location 115 for particulates (per step 314).

If in step 316 dynamic cleaning program 122 determines that a particulate is detected, and if the detected particulate exceeds a given conditional threshold, then in step 318, dynamic cleaning program 122 determines and/or instructs secondary instrument to perform one or more remedies, to eliminate and/or reduce the detected particulate.

Thereafter, in step 320, dynamic cleaning program 122 resets the detected condition. This allows dynamic cleaning program 122 in step 322 to determine if the remedy performed was successful in reducing the particulate to an allowed level, by monitoring the environment (similar to step 314). If dynamic cleaning program 122 determines that the remedy performed was successful, then in step 324 the system is reset and dynamic cleaning program 122 will continue to monitor the area for particulates (per step 314).

If however, dynamic cleaning program 122 determines that the remedy performed was not successful, then in step 326 dynamic cleaning program 122 sends an alert to a user, notifying the user to manually take corrective actions. For example, the alert may include a generated display that can be manipulated on the fly, and updated according to user actions/selections. For example, a user may select “dust apartment” and then 122 can “predict” or update the display to show a 20% reduction in allergen in response to the user selection of “dusting”. In another example, the alert may trigger augmented reality which will pinpoint on an overlay of the location as the cause of the issue. This allows a user to precisely take corrective actions saving time and guesswork. Upon receiving an indication that corrective action was taken, then in step 328 the system is reset and dynamic cleaning program 122 will continue to monitor the area for particulates (per step 314).

FIG. 4 is a block diagram of internal and external components of a computer system 400, which is representative of the computer systems of FIG. 1, in accordance with an embodiment of the present invention. It should be appreciated that FIG. 4 provides only an illustration of one implementation, and does not imply any limitations with regard to the environments in which different embodiments may be implemented. Many modifications to the depicted environment may be made.

Computer system 400 includes communications fabric 402, which provides communications between computer processor(s) 404, memory 406, persistent storage 408, communications unit 412, and input/output (I/O) interface(s) 414. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses.

Memory 406 and persistent storage 408 are computer readable storage media. In this embodiment, memory 406 includes random access memory (RAM) 416 and cache memory 418. In general, memory 406 can include any suitable volatile or non-volatile computer readable storage media.

Persistent storage 408 may include, for example, a plurality of magnetic hard disk drives. Programs are stored in persistent storage 408 for execution and/or access by one or more of the respective computer processors 404 via one or more memories of memory 406. In this embodiment, persistent storage 408 includes a magnetic hard disk drive. Alternatively, or in addition to a magnetic hard disk drive, persistent storage 408 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 412, in these examples, provides for communications with other data processing systems or devices. In these examples, communications unit 412 includes one or more network interface cards. Communications unit 412 may provide communications through the use of either or both physical and wireless communications links. Software and data used to practice embodiments of the present invention can be downloaded to computer system 400 through communications unit 412 (i.e., via the Internet, a local area network, or other wide area network). From communications unit 412, the software and data may be loaded to persistent storage 408.

I/O interface(s) 414 allows for input and output of data with other devices that may be connected to computer system 400. For example, I/O interface 414 may provide a connection to external devices 420, such as a keyboard, keypad, a touch screen, and/or some other suitable input device. External devices 420 can also include portable computer readable storage media such as, for example, thumb drives, portable optical or magnetic disks, and memory cards. Software and data used to practice embodiments of the present invention, can be stored on such portable computer readable storage media and can be loaded onto persistent storage 408 via I/O interface(s) 414. I/O interface(s) 414 also connect to a display 422.

Display 422 provides a mechanism to display data to a user and may be, for example, a computer monitor. Display 422 can also be an incorporated display and may function as a touch screen, such as a built-in display of a tablet computer.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A method comprising:
receiving, by one or more processors, a set of information from the at least one sensor, wherein the set of information comprise environmental readings;
detecting, by one or more processors, the environmental readings which exceed a threshold;
responsive to detecting the environmental readings have exceeded the threshold, determining, by one or more processors, a remedy to bring the set of information below the threshold; and
executing, by one or more processors, the determined remedy.

2. The method of claim 1, further comprising:
identifying, by one or more processors, an area of concern, wherein the area of concern comprises at least one of:
a location where insects can enter,
a location where rodents can enter,
a location where mold can grow, and
a location where allergens can spread.

3. The method of claim 1, further comprising:
deriving, by one or more processors, a pattern, based on the set of information, wherein the pattern identifies one or more common precursors to the environmental readings exceeding a threshold.

4. The method of claim 3, further comprising:
responsive to receiving the set of information, detecting, by one or more processors, the one or more common precursors, wherein the one or more common precursors identify an environmental reading which will cause the threshold to be exceeded; and
determining, by one or more processors, a remedy to prevent the one or more common precursors from exceeding the threshold.

5. The method of claim 3, further comprising:
responsive to deriving a pattern, adjusting, by one or more processors, a threshold for an area of concern.

6. The method of claim 1, wherein the determining remedy comprises:
deploying, by one or more processors, one or more devices, wherein each device performs at least one remedy to a designated area.

7. The method of claim 6, wherein the at least one remedy is at least one of:
closing air gaps;
spraying insecticide;
spraying mold inhibitor; and
providing a close inspection of the designated area.

8. The method of claim 1, further comprising:
depicting, by one or more processors, an augmented reality display to a user to visualize an area where either insects can enter, mold can grow, and allergens can spread.

9. The method of claim 1, further comprising:
responsive to receiving the set of information from the at least one sensor, adjusting, by one or more processors, the threshold, wherein adjusting the threshold increases and decreases based on one or more environmental readings.

10. The method of claim 1, further comprising:
responsive to executing the determined remedy, identifying, by one or more processors, that the remedy failed to reduce the one or more environmental readings below the threshold; and
notifying, by one or more processors, a user.

11. The method of claim 1, wherein executing the determining remedy comprises:
utilizing, by one or more processors, a heating ventilation and air-conditioning (HVAC) system.

12. The method of claim 11, wherein utilizing the HVAC system comprises at least one of:
increasing internal pressure of a building;
activating one or more specialty filters;
exhausting existing air within building;
increasing humidity;
decreasing humidity; and
increasing the circulation of air within the building.

13. A computer program product comprising:
a computer readable storage medium and program instructions stored on the computer readable storage medium, the program instructions comprising:
program instructions to receive a set of information from the at least one sensor, wherein the set of information comprise environmental readings;
program instructions to detect the environmental readings which exceed a threshold;
responsive to detecting the environmental readings have exceeded the threshold, program instructions to determine a remedy to bring the set of information below the threshold; and
program instructions to execute the determined remedy.

14. The computer program product of claim 13, further comprising:
program instructions to identify an area of concern, wherein the area of concern comprises at least one of:
a location where insects can enter,
a location where rodents can enter,
a location where mold can grow, and
a location where allergens can spread.

15. The computer program product of claim 13, wherein the determining remedy comprises:
program instructions to deploy one or more devices, wherein each device performs at least one remedy to a designated area.

16. The computer program product of claim 15, wherein the at least one remedy is at least one of:
program instructions to close air gaps;
program instructions to spray insecticide;
program instructions to spay mold inhibitor; and
program instructions to provide a close inspection of the designated area.

17. The computer program product of claim 13, wherein executing the determining remedy comprises:
program instructions to utilize a heating ventilation and air-conditioning (HVAC) system.

18. A computer system comprising:
one or more computer processors;
one or more computer readable storage media;
program instructions stored on the one or more computer readable storage media for execution by at least one of the one or more processors, the program instructions comprising:
program instructions to receive a set of information from the at least one sensor, wherein the set of information comprise environmental readings;
program instructions to detect the environmental readings which exceed a threshold;
responsive to detecting the environmental readings have exceeded the threshold, program instructions to determine a remedy to bring the set of information below the threshold; and
program instructions to execute the determined remedy.

19. The computer system of claim 18, further comprising:
program instructions to identify an area of concern, wherein the area of concern comprises at least one of:
a location where insects can enter,
a location where rodents can enter,
a location where mold can grow, and
a location where allergens can spread.

20. The computer system of claim 18, wherein executing the determining remedy comprises:
program instructions to utilize a heating ventilation and air-conditioning (HVAC) system.
